SEQUENCE LISTING (7 sequences)

SEQ ID NO: 1 - DNA, 20 bp, Artificial Sequence
  Synthetic
ttacgtccctgccctttgta

SEQ ID NO: 2 - DNA, 17 bp, Artificial Sequence
  Synthetic
ggttggtttcttttcct

SEQ ID NO: 3 - DNA, 17 bp, Artificial Sequence
  Synthetic
tcctgagggaaacttcg

SEQ ID NO: 4 - DNA, 17 bp, Artificial Sequence
  Synthetic
acccgctgaacttaagc

SEQ ID NO: 5 - DNA, 643 bp, S. sanghuang
  misc_feature: ITS sequence
gtgctggtgcgaaatcgcgcatgtgcacggtcttcgcgctcaaatccaactcaaacccctgtgcaccttatatatcgcgagtcgaagttagtagcctgaggtcttgtaagtaattagtagaagggcgaaagcgcgactcttgctcgttaggtagcctttcgaaaatgaaagcgagtgcgtcgggtgaagacttcggcttgtcgttacaaaacaccttatattgtctttgtgaatgtaatgctccttgtgggcgaaaataaatacaactttcaacaacggatctcttggctctcgcatcgatgaagaacgcagcgaaatgcgataagtaatgtgaattgcagaattcagtgaatcatcgaatctttgaacgcaccttgcgccccttggtattccgaggggcatgcctgtttgagtgtcatgtttatctcaaaccgctcgtctttcttaattgaagggcttgaggtttggacttggaggtttactgctggcgcctttcgaggggtcggctcctcttaaatacattagctgggctttggctcgcgtttacggtgtaatagttgattccattcaccaacgagcgcttgcctgacgagcttgcttctagccgtccgcgtcgtcggacaaggagtcacctccttcttga

SEQ ID NO: 6 - DNA, 877 bp, S. sanghuang
  misc_feature: LSU sequence
ctgcgagtgaagcgggaagagctcaaatttaaaatctggcggccttctggacgtccgagttgtagtctggagaagtgttatccgcgtcggaccgtgtacaagtctcctggaacggagcgtcatagagggtgagaatcccgtccatgacacggacgcccgatgctatgtgaggcactctcgaagagtcgagttgtttgggaatgcagctcaaaatgggtggtaaattccatctaaagctaaatattggcgagagaccgatagcgaacaagtaccgtgagggaaagatgaaaagcactttggaaagagagttaaacagtacgtgaaattgttgaaagggaaacgcttgaagtcagtcgcgtcccgtggaactcagcctggtttcgacctggtgtactttccatgtggacgggtcaacatcaatttcggccggtggacaagggcgaggggaatgtagcgttgcttcggcgacgtgttatagccccccgtcgcatacactggctgggattgaggaccgcagcacgcccttgtggccggggggttcgccccacgtaacgtgcttaggatgttggcataatggctttaagcgacccgtcttgaaacacggaccaaggagtctaacatgcttgcgagtgttcgggtggaaaacccttgcgcgtaatgaaagtgaaagttgggaacctccgcgagggggtgcaccgacgcccggccctgacgttctctgacggtgccgcggtagagcacgtatgttgggacccgaaagatggtgaactatgcctgaatagggcgaagccagaggaaactctggtggaggctcgtagcgattctgacgtgcaaatcgatcgtcaaatttgggtataggggcgaaagactaatcgaa

SEQ ID NO: 7 - DNA, 13 bp, Artificial Sequence
  Synthetic estrogen response elements (EREs)
  misc_feature (6)..(8): n is a, c, g, or t
ggtcannntgacc

(19) United States
(12) Patent Application Publication
(10) Pub. No.: US 2020/0147158 A1
(43) Pub. Date: May 14, 2020

(54) SANGHUANGPORUS SANGHUANG STRAINS AND THEIR PRODUCTS, EXTRACTS AND APPLICATIONS

(71) Applicants: FOOD INDUSTRY RESEARCH AND DEVELOPMENT INSTITUTE, HSINCHU CITY (TW); NATIONAL MUSEUM OF NATURAL SCIENCE, Taichung City (TW)

(72) Inventors: Sue-Fan Wu, Hsinchu City (TW); Ta-Wei Liu, Hsinchu City (TW); Ming-Der Wu, Hsinchu City (TW); I-Ching Chen, Hsinchu City (TW); Sheng-Hua Wu, Taichung City (TW); Sung-Yuan Hsieh, Hsinchu City (TW); Hing-Yuen Chan, Hsinchu City (TW); Gwo-Fang Yuan, Hsichu City (TW); Ming-Jen Cheng, Hsinchu City (TW)

(73) Assignees: FOOD INDUSTRY RESEARCH AND DEVELOPMENT INSTITUTE, HSINCHU CITY (TW); NATIONAL MUSEUM OF NATURAL SCIENCE, Taichung City (TW)

(21) Appl. No.: 16/185,387

(22) Filed: Nov. 9, 2018

(51) Int. Cl. A61K 36/07; A61P 5/30; C12Q 1/02; A61P 15/00; A61K 31/57; A61K 31/566

(52) U.S. Cl. CPC A61K 36/07; A61P 5/30; A61K 31/566; A61P 15/00; A61K 31/57; C12Q 1/025

(57) ABSTRACT

The present invention relates to the preparation of a liquid fermentate of Sanghuangporus or an extract of Sanghuangporus, compounds identified from the liquid fermentate or the extract, and their novel activity.

### FIELD OF THE INVENTION

The present invention relates to a field of microorganism in the treatment or prevention of estrogen-dependent condition. Particularly, the present invention provides Sanghuangporus sanghuang strains, the preparation of an extract of the Sanghuangporus sanghuang strains, compounds identified from the extract, and the novel activity of the compounds.

### BACKGROUND OF THE INVENTION

Estrogen receptor (“ER”) is a ligand-activated transcriptional regulatory protein that mediates induction of a variety of biological effects through its interaction with endogenous estrogens. ER has been found to have two isoforms, ERα (alpha) and ERβ (beta). Said two isoforms have been found to distribute in different tissues. ERα mainly distributes in breast, ovary, and uterus; while ERβ distributes in bone, lung, endothelial cells, prostate, and other tissues. Both isoforms have high affinity to estrogen, but are significant different in affinity to certain analogues from other sources. Therefore, the so-called Selective Estrogen. Receptor Modulator (SERMs) may be existed. SERMs are structurally and functionally similar to estrogen, but their responses to different estrogen receptors are different, and thus are able to regulate different ERs in different organs. Ideal SERMs should have an estrogen-like effect so that they can provide antagonism in mammary glands, uterus, etc., and positive regulation erect in cardiovascular systems, bones, and central nervous systems. These differences make SERMs capable of causing differential. physiological impact in different tissues (Paterni, I., Granchi, C., Katzenellenbogen, J. A., Minutolo, F., (2014) Estrogen receptors alpha (ERα) and beta (ERβ): subtype-selective ligands and clinical potential. Steroids. pii S0039-128X (14), 00151-00152.).

Mushrooms (macrofungi) have been accumulated and valued as traditional sources of natural bioactive secondary metabolites for many centuries. Medicinal mushrooms such as Ganoderma lucidum, Phellinus linteus, and Trametes versicolor, have an established history of use in traditional Asian treatments, and many novel biologically active compounds have been reported, While some medicinal species have been well investigated, many species remain chemically unexplored and poorly investigated.

Sanghuangporus sanghuang is distributed in mainland China, Japan, Korea, Myamnar and Taiwan, only grows on Morus trees, and is very rare in the wild, S. sanghuang and other species in this genus still remain chemically unexplored.

### SUMMARY OF THE INVENTION

One aspect of the invention is to provide a method for the preparation of a liquid fermentate of Sanghuangporus or a Sanghuangporus extract.

Another aspect of the invention is to provide a method for the preparation of

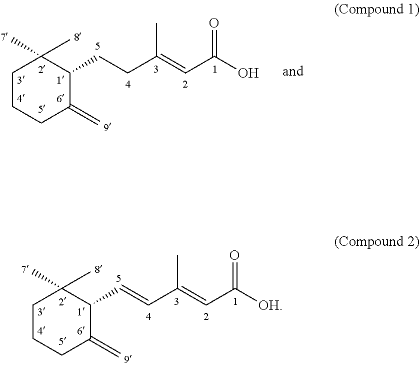

Another aspect of the invention is to provide a liquid fermentate of Sanghuangporus or a Sanghuangporus extract obtainable from the method of the invention.

Another aspect of the invention is to provide a pharmaceutical composition comprising the liquid fermentate of Sanghuangporus or Sanghuangporus extract of the invention and a pharmaceutically acceptable carrier.

Another aspect of the invention is to provide a method for preventing or treating an estrogen-dependent condition, disease, disorder, or syndrome of a subject in need thereof, comprising administering to the subject the extract or the pharmaceutical composition of the invention.

Another aspect of the invention is to provide a method for preventing or treating an estrogen-dependent condition, disease, disorder, or syndrome in a subject in need thereof, comprising administering to the subject a therapeutically effective amount of the liquid fermentate of the invention.

Another aspect of the invention is to provide a method for preventing or treating an estrogen-dependent condition, disease, disorder, or syndrome in a subject in need thereof, comprising administering to the subject a therapeutically effective amount of a compound selected from Compound 1 or Compound 2.

Another aspect of the invention is to provide a method for screening a compound having an ER activity comprising the steps of.

(a) providing transfected cells expressing an estrogen response elements (ERE) and a reporter gene;

(b) culturing the transfected cells in the presence of the compound, a positive control or a negative control; and

(c) measuring the activity of the reporter gene;
wherein the elevated activity of the reporter gene as compared to the negative control indicates that the compound has an ER activity, and wherein the positive control is Compound 1 or Compound 2 as defined above.

Another aspect of the invention is to provide a S. sanghuang 38847, which was deposited with the Deutsche Sammlung von Mikroorganismen and Zellkulturen (DSMZ) on 29 Aug. 2018 in accordance with the Budapest Treaty, and assigned the Accession No. DSM 32914.

Still another aspect of the invention is to provide the use the liquid fermentate of Sanghuangporus or the extract or the pharmaceutical composition of the invention in the manufacture of a medicament for preventing or treating an estrogen-dependent condition, disease, disorder, or syndrome.

Still another aspect of the invention is to provide the use of Compound 1 or Compound 2 as defined above in the manufacture of a medicament for preventing or treating an estrogen-dependent condition, disease, disorder, or syndrome,

The present invention is described in detail in the following sections. Other characterizations, purposes and advantages of the present invention can be easily found in the detailed descriptions and claims of the invention.

### BRIEF DESCRIPTION OF THE DRAWINGS

FIG. 1 shows the ER activity of Compounds 1 and 2 of the invention.

### DETAILED DESCRIPTION OF THE INVENTION

### Definitions

Unless otherwise defined herein, scientific and technical terms used in connection with the present invention shall have the meaning commonly understood by those of ordinary skill in the art. The meaning and scope of the terms should be clear; however, in the event of any latent ambiguity, definitions provided herein take precedence over any dictionary or extrinsic definition.

Unless otherwise required by context, singular terms shall include the plural and plural terms shall include the singular. For example, the term “a” or “an,” as used herein, is defined as one or more than one.

The term “or” in the claims is used to mean “and/or” unless explicitly indicated to refer to alternatives only or the alternatives are mutually exclusive.

Ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be, understood that the particular value forms another embodiment. It will be further understood that the endpoints of each of the ranges are significant both in relation to and independently of the other endpoint. As used herein the term “about” refers to ±30%, preferably ±20%, more preferably ±10%, and even more preferable ±5%.

As used herein, the term “Sanghuangporus” refers to any species of the Sanghuangporus genus.

As used herein, the term “secondary metabolite” refers to a compound, derived from primary metabolites, that is produced by fungi, is not a primary metabolite and is not required for growth.

As used herein, the term “extract” refers to all possible extracts that are obtained during the sample preparation process and comprise an active (lead) compound(s). The extract may be in the form of a liquid, solid or powder.

As used herein, the term “active extract” refers to all possible extracts that show the desired bioactivity Examples of such active extracts of the invention include, but are not limited to, crude extracts, liquid fermentate, column chromatographic fractions, High Performance Liquid Chromatography (HPLC)-purified fractions, thin layer chromatography(TLC)-purified fractions, etc.

As used herein, the term “solvent” refers o a carbon-based liquid capable of dissolving another substance.

As used herein, the term “non-polar solvent” refers to, any organic solvents with a polarity index of not greater than about 2.0. Examples of such non-polar solvents include, but are not limited to, hexane, petroleum ether, carbon tetrachloride, and a mixture thereof.

As used herein, the term “polar solvent” refers to any organic solvents with a polarity index of greater than about 2.0, and generally easily miscible with water. Examples of such moderately polar solvent include, but are not limited to, methanol ethanol, acetonitrile, and a mixture thereof.

As used herein, the term “silica gels” refers to a granular, vitreous, porous form of silicon dioxide made synthetically from sodium silicate. Silica gel contains a nano-porous silica micro-structure, suspended inside of a liquid.

As used herein, the term “elution solution” as used herein refers to the solution that is used to elute the extract from the column chromatography, ion exchange resin, etc.

As used herein, the term “preventing” refers to delaying the onset of symptoms of a susceptible subject, reducing the occurrence of a disorder or condition, or inhibiting the occurrence of the disorder or condition, or arresting the development of the disorder or condition.

As used herein, the term “treating” or “treatment” refers to alleviating, relieving, reversing and/or improving a disorder or condition or one or more symptoms thereof, or stopping the symptoms of the disease or condition in a susceptible subject.

As used herein, the term “subject” refers to animals, especially mammals. in one preferred embodiment, the term subject denotes “humans.”

As used herein, the term “therapeutically effective amount” refers to the amount of an active ingredient used alone or in combination with other treatments/medicaments for preventing or treating periodontitis that shows therapeutic efficacy,

As used herein, the term “pharmaceutically acceptable carrier” refers to solvents, diluents, binders, adhesives, adjuvants, excipients, acceptors, stabilizer, analogues, flavoring agents, sweetening agents, emulsifying agents or preservative agents, which are well known to persons of ordinary skill in the art, for manufacturing pharmaceutical or dietary compositions. in Examples of pharmaceutically acceptable carriers include, but are not limited to, water, saline, buffers, and inert, nontoxic solids.

As used herein, the term “administering” or “administration” refers to the methods that may be used to enable delivery of the composition or medicament of the present invention to the desired site of biological action.

As used herein, the terms “condition,” “disease,” “disorder,” or “syndrome” may be used interchangeably

### Sources of Sanghuangporus

Examples of Sanghuangporus species include, but are not limited to Sanghuangporus microcystideus, Sanghuangporus zonatus, Sanghuangporus baumii, Sanghuangporus sanghuang, Sanghuangporus lonicericola, Sanghuangporus vaninii, Sanghuangporus weirianus, Sanghuangporus alpinus and Sanghuangporus weigelae.

In a preferred embodiment of the invention, the Sanghuangporus is Sanghuangporus sanghuang.

### The Preparation Processes and Extracts Obtainable Therefrom

The invention provides a method of culturing the basidiomycete Sanghuangporus sanghuang for the production of diverse secondary metabolites.

The invention provides a method for preparing a liquid fermentate of Sanghuangporus sanghuang, comprising culturing Sanghuangporus in a broth under a condition suitable for Sanghuangporus.

The invention provides a method for the preparation of a Sanghuangporus extract, comprising the steps of:

(a) obtaining a liquid fermentate of Sanghuangporus by culturing Sanghuangporus in a broth; and

(b) obtaining the Sanghuangporus extract by mixing the liquid fermentate of Sanghuangporus with alcohols.

The invention provides a method for the preparation of

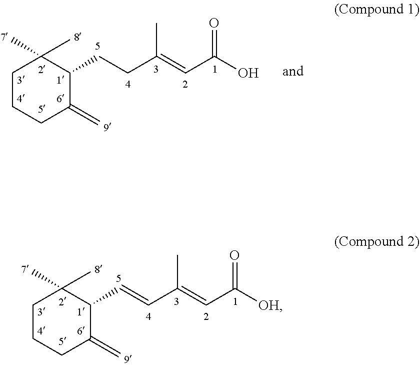

comprising the steps of:

(a) obtaining a liquid fermentate of Sanghuangporus by culturing Sanghuangporus in a broth under a condition suitable for Sanghuangporus;

(b) obtaining an alcohol-soluble extract by mixing the liquid fermentate of Sanghuangporus with alcohols;

(c) subjecting the alcohol-soluble extract to silica gel column chromatography;

(d) obtaining different eluates by eluting the column with elution solutions; and

(e) collecting a first eluted fraction and a second eluted fraction by thin layer chromatography (CH₂Cl₂:acetone: 40:1), wherein the first eluted fraction comprises the Compound 1 and the second eluted fraction comprises the Compound 2.

In step (b) of the present invention, the alcohols are methanol, ethanol or n-butanol.

In step (d) of the present invention, the column is dined with CH₂Cl₂/ethyl acetate (1:1).

In step (d) of the present invention, the column ^{is} eluted with 2×-, 3×-, 4×- or 5×-column volume CH₂Cl₂/ethyl acetate (1:1).

In step (d) of the present invention, the column is sequentially eluted with CH₂Cl₂/ethyl acetate (1:1), CH₂Cl₂/ethyl acetate/MeOH (1:1:0.5) and CH₂Cl₂/ethyl acetate/MeOH (1:1:1).

In step (d) of the present invention, the column is sequentially elated with 2×-, 3×-, 4×- or 5×- column volume CH₂Cl₂/ethyl acetate (1:1), 2×-, 3×-, 4×- or 5×-column volume CH₂Cl₂/ethyl acetate/MeOH (1:1:0.5) and 2×-, 3×-, 4×- or 5×-column volume CH₂Cl₂/ethyl acetate/MeOH (1:1).

In one embodiment, after step (d), which further comprises a step of concentrating the doted fraction and/or drying the fraction to obtain a paste or solid fermentate.

In step (e) of the present invention, the elution buffer is CH₂Cl₂:acetone (10:1), CH₂Cl₂:acetone (20:1), CH₂Cl₂:acetone (30:1) , CH₂Cl₂:acetone (40:1) CH₂Cl₂:acetone (50:1) or CH₂Cl₂:acetone (60:1).

In some embodiments, solvents are non-polar solvents or polar solvents. According to the present invention, the silica gel used can be Silica Gel 60 GF254, Silica Gel 60 (less than 0.063 mm), Silica Gel 60 (0.2-0.5 mm), Silica Gel 60 (0.063-0.200 mm), Silica Gel 60 extra pure, Silica Gel 60 (0.040-0.063 mm), Silica Gel 60 (35-70 mm), or Silica Gel 60 F254 (0.063-0.200 mm).

According to the invention, the elution solutions used in the column chromatography may include, but are not limited to, CH₂Cl₂/ethyl acetate, CH₂Cl₂/ethyl acetate/MeOH, hexanelethyl acetate, CH₂Cl₂:acetone, methanol, ethanol and ethanol/ethyl acetate.

In an embodiment of, the invention, the volume ratio between CH₂Cl₂and ethyl acetate in the CH₂Cl₂/ethyl acetate solvent, the volume ratio between n-hexane and ethyl acetate in the n-hexane/ethyl acetate solvent and the volume ratio between ethanol and ethyl acetate in the ethanol/ethyl acetate solvent may be 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, and 95:5. in a preferred embodiment of the invention., the ratio of the CH₂Cl₂/ethyl acetate solvent used is 50:50.

In an embodiment of the invention, the volume ratio of CH₂Cl₂/ethyl acetate/MeOH may be 1:1:0.3, 1:1:0,4, 1:1:0,6, 1:110,7, 1:1:0.8, 1:1:0.9, 1:1:1, 1:0,3:1, 1:0,4:1, 1:0,5:1, 1:0.6:1, 1:0,7:1, 1:0.8:1, 1:0.9:1, 0.1:1:1, 0.4:1:1, 0.5:1:1, 0,6:1:1, 0.7:1:1, 0.8:1:1 or 0.9:1:1.

The present invention also provides a liquid fermentate of Sanghuangporus or Sanghuangporus extracts obtained from the processes described above.

In an embodiment of the invention, the fermentate of Sanghuangporus or Sanghuangporus extract comprises

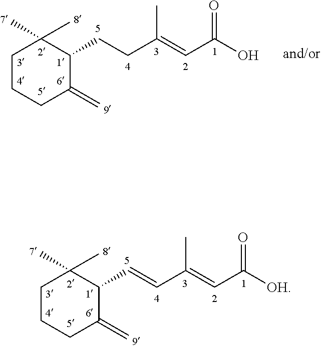

The present invention also provides composition comprising compound 1

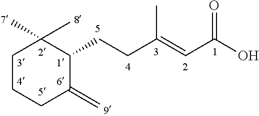

and compound 2.

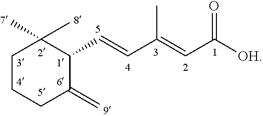

### Culture Condition for Sanghuangporus

According to one embodiment, S. sanghuang is cultured on malt extract agar (MEA) medium for 6 days, 7 days, 8 days, 9 days, 10 days, 11 days, 12 days, 13 days, 14 days, 15 days, 16 days, 17 days. 18 days, 19 days, 20 days, 21 days, 22 days, 23 days. 24 days, 25 days, 26 days, 27 days, 28 days, 29 days or 30 days.

S. sanghuang colonies (6- to 21-Day) is cultured in a cultured in a flask containing a liquid cultural medium at 22° C., 23° C., 24° C., 25° C., 26° C., 27° C., 28° C., 29° C. or 30° C. for 7 days, 8 days, 9 days, 10 days, 11 days, 12 days, 13 days, 14 days, 15 days, 16 days, 17 days, 18 days, 19 days, 20 days or 21 days.

The liquid cultural medium comprises corn starch, corn steep liquor, yeast extract, sea salt distilled water at pH about 5.8-6.3.

### ER Platform

The invention provides a method for screening a compound having an estrogen receptor (ER) activity comprising the steps of:

(a) providing transfected cells expressing an estrogen response elements (ERE) and a reporter gene;

(b) culturing the transfected cells in the presence of the compound, a positive control or a negative control; and

(c) measuring the activity of the reporter gene;
wherein the elevated activity of the reporter gene as compared to the negative control indicates that the compound has an ER activity, and wherein the positive control is

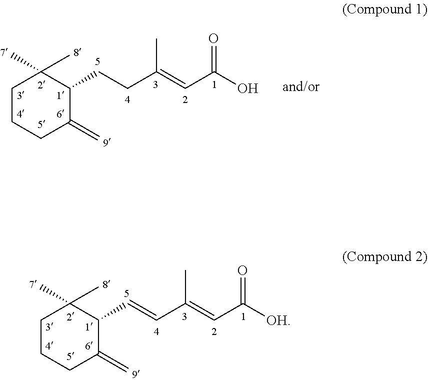

In an embodiment of the invention, EREs are 5′-GGTCAnnnTGACC-3′ (wherein n is A, T, C or G) (SEQ ID NO: 7).

In an embodiment of the invention, the reporter gene may be alkaline phosphatase (SEAP), β-galactosidase, chloramphenicol acetyltransferase, green fluorescent protein (GFP) or red fluorescent protein (RFP).

### Compositions

According to one embodiment, the invention provides a composition comprising a therapeutically effective amount of a Sanghuangporus extract obtainable from the preparation method of the present invention and a pharmaceutically acceptable carrier.

Oral compositions generally include an inert diluent or an edible carrier. Oral compositions can be liquid, or can be enclosed in gelatin capsules or compressed into tablets, Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. Trans mucosal administration can be accomplished through the use of nasal sprays or suppositories. For transdermal administration, the active compounds typically are formulated into ointments, salves, gels, or creams as generally known in the art.

The composition can be administered to a patient orally or parenterally in the conventional forms of preparations, such as capsules, microcapsules, tablets, granules, powder, troches, pills, suppositories, injections, suspensions and syrups. Suitable formulations can be prepared by methods commonly employed using conventional, organic or inorganic carriers, such as an excipient sucrose, starch, mannitol, sorbitol, lactose, glucose, cellulose, talc, calcium phosphate or calcium carbonate), a binder (e.g., cellulose, methylcellulose, hydroxymethylcelludose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellutose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). In some embodiments, the composition of the present invention can be in the form of a semi-solid or solid such as a toothpaste, a gel dentifrice, a dental powder, a denture cleansing tablet, a chewing gum, or a solid lozenge or the like.

### Utility

The extract, crude extract, liquid fermentate and composition of the present invention can be used to prevent, treat or reduce the risk of an estrogen-dependent condition, disease, disorder, or syndrome in a subject in need thereof. Therefore, the present invention provides a method for preventing or treating an estrogen-dependent condition, disease, disorder, or syndrome in a subject in need thereof, which comprises administering to the subject the extract, crude extract, liquid fermentate and composition. of the present invention.

In a preferred embodiment, the estrogen-dependent condition, disease, disorder, or syndrome includes, but is not limited to mastodynia (breast pain tenderness), breast fibroids, mammoplasia (breast enlargement), macromastia (breast hypertrophy), cardiovascular disease, stroke, gynecomastia, breast cancer, osteoporosis, precocious puberty in girls, melasma, menorrhagia, endometriosis, endometrial hyperplasia, adenomyosis, uterine fibroids, uterine cancers (e.g., endometrial cancer), ovarian cancer, and hyperestrogenism in males such as in certain conditions like cirrhosis and Klinefelter's syndrome.

In a preferred embodiment, the cardiovascular disease includes, but is not limited to hypertension (high blood pressure), coronary heart disease (heart attack), cerebrovascular disease (stroke), peripheral vascular disease, heart failure, rheumatic heart disease, congenital heart disease, cardiomyopathies, hypertensive heart disease, rheumatic heart disease, cardiomyopathy, heart arrhythmia, valvular heart disease, carditis, aortic aneurysms, thromboembolic disease, and venous thrombosis.

In a preferred embodiment, the composition optionally comprises a conventional drug or agent useful in the prevention or treatment of an estrogen-dependent condition, disease, disorder, or syndrome. The normal dosages of these conventional drugs or agents are well known in the art. These conventional drugs or agents include, but are not limited to SERMs (such as clomifene, ormeloxifene, raloxifene, tamoxifen, toremifene, lasofoxifene and ospemifene), estrogen receptor antagonists such as fulvestrant, aromatase inhibitors such as anastrozole and exemestane, gonadotropin-releasing hormone (GnRH) analogues such as leuprorelin and cetrorelix, and/or other antigonadotropins such as danazol, gestrinone, megestrol acetate, and medroxyprogesterone acetate.

In a preferred embodiment, the composition optionally comprises a conventional drug or agent useful in the prevention or treatment of osteoporosis. The normal dosages of these conventional drugs or agents are well known in the art. These conventional drugs or agents include, but are not limited to an antiresoptive agent (e.g. bisphosphonate, receptor activator of nuclear factor kappa-B ligand (RANKL) inhibitor, SERMs (such as clomifene, ormeloxifene, raloxifene, tamoxifen, toremifene, lasofoxifene and ospemifene), anabolic agent (e.g. teriparatide) and strontium ranelate.

In a preferred embodiment, the composition optionally comprises a conventional drug or agent useful in the prevention or treatment of breast cancer. The normal dosages of these conventional drugs or agents are well known in the art. These conventional drugs or agents include, but are not limited to Abemaciclib, Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Cyclophosphamide, Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole). Fluorouracil Injection, Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisquali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Ribocic lib, Tamoxifen Citrate, Taxol (Paclitaxel), Taxotere (Docetaxel), Thiotepa, Toremifene, Trastuzumab, Trexall (Methotrexate), Tykerb (Lapatinib Ditosylate) Verzenio (Abemaciclib), Vinblastine Sulfate, Xeloda (Capecitabine), Zoladex (Goserelin Acetate).

The following examples are provided to aid those skilled in the art in practicing the present invention. Even so, the examples should not be construed to unduly limit the present invention as modifications to and variations on the embodiments discussed herein may be made by those having ordinary skill in the art without departing from the spirit or scope of the present inventive discovery.

### Example 1

### Microbial Materials

The fungi used in this study were isolated from the leaves of Morus sp., and identified as Sanghuangporus sanghuang (S. sanghuang) on the basis of the rDNA internal transcribed spacer (ITS) sequence and the ribosomal large subunit (ESU) sequence.

Identification

The rDNA ITS sequence and LSU sequence from fungi strains were analyzed. The primer sequences used were shown in Table 1 below.

TABLE 1

| Primer | | |
|---|---|---|
| Name | Sequence | SEQ ID NO |
| V9G | 5′-TTACGTCCCTGCCCTTTGTA-3′ | SEQ ID NO: 1 |
| LR1 | 5′-GGTTGGTTTCTTTTCCT-3′ | SEQ ID NO: 2 |
| LR5 | 5′-TCCTGAGGGAAACTTCG-3′ | SEQ ID NO: 3 |
| LROR | 5′-ACCCGCTGAACTTAAGC-3′ | SEQ ID NO: 4 |

The PCR reaction was conducted using V9G/LR1 primer set or LR5/LROR primer set with the condition: (1) 94° C., 5 minutes for 1 cycle; (2) 94° C., 30 seconds; 50° C., 1 minute; and 72° C. 1 minute for 35 cycles; and (3) 72° C., 10 minutes for 1 cycle.

The segment ITS1-5.8S-ITS2 (643 bp) was amplified. Upon comparison, it was found that the segment ITS1-5.8S-ITS2 has a sequence similarity of 99% ( 639/642) with the type strain Wu0903-1 (Accession No. JN794061) in the NCBI GenBank database.

The segment LSU (877 bp) was amplified. Upon comparison, it was found that there are two mutations (M=A or C, Y is T or C) and the segment LSU has a coverage rate of 84.6% ( 720/851) and a sequence similarity of 99% ( 718/720) with the strain Wu0903-1.

S. sanghuang 38847 has the following ITS sequence and LSU sequence:

ITS sequence (SEQ ID NO: 5):
gtgctggtgcgaaatcgcgcatgtgcacggtcttcgcgctcaaatccaac
tcaaacccctgtgcaccttatatatcgcgagtcgaagttagtagcctgag
gtcttgtaagtaattagtagaagggcgaaagcgcgactcttgctcgttag
gtagcctttcgaaaatgaaagcgagtgcgtcgggtgaagacttcggcttg
tcgttacaaaacaccttatattgtctttgtgaatgtaatgctccttgtgg
gcgaaaataaatacaactttcaacaacggatctcttggctctcgcatcga
tgaagaacgcagcgaaatgcgataagtaatgtgaattgcagaattcagtg
aatcatcgaatctttgaacgcaccttgcgccccttggtattccgaggggc
atgcctgtttgagtgtcatgtttatctcaaaccgctcgtctttcttaatt
gaagggcttgaggtttggacttggaggtttactgctggcgcctttcgagg
ggtcggctcctcttaaatacattagctgggctttggctcgcgtttacggt
gtaatagttgattccattcaccaacgagcgcttgcctgacgagcttgctt
ctagccgtccgcgtcgtcggacaaggagtcacctccttcttga
LSU sequence (SEQ ID NO: 6):
ctgcgagtgaagcgggaagagctcaaatttaaaatctggcggccttctgg
acgtccgagttgtagtctggagaagtgttatccgcgtcggaccgtgtaca
agtctcctggaacggagcgtcatagagggtgagaatcccgtccatgacac
ggacgcccgatgctatgtgaggcactctcgaagagtcgagttgtttggga
atgcagctcaaaatgggtggtaaattccatctaaagctaaatattggcga
gagaccgatagcgaacaagtaccgtgagggaaagatgaaaagcactttgg
aaagagagttaaacagtacgtgaaattgttgaaagggaaacgcttgaagt
cagtcgcgtcccgtggaactcagcctggtttcgacctggtgtactttcca
tgtggacgggtcaacatcaatttcggccggtggacaagggcgaggggaat
gtagcgttgcttcggcgacgtgttatagccccccgtcgcatacactggct
gggattgaggaccgcagcacgcccttgtggccggggggttcgccccacgt
aacgtgcttaggatgttggcataatggctttaagcgacccgtcttgaaac
acggaccaaggagtctaacatgcttgcgagtgttcgggtggaaaaccctt
gcgcgtaatgaaagtgaaagttgggaacctccgcgagggggtgcaccgac
gcccggccctgacgttctctgacggtgccgcggtagagcacgtatgttgg
gacccgaaagatggtgaactatgcctgaatagggcgaagccagaggaaac
tctggtggaggctcgtagcgattctgacgtgcaaatcgatcgtcaaattt
gggtataggggcgaaagactaatcgaa

S. sanghuang 38847 was deposited with the Deutsche Sammlung von Mikroorganismen und Zellkulturen (DSMZ) on 29 Aug. 2018 in accordance with the Budapest Treaty, and assigned the Accession No. DSM 32914.

### Example 2

### Preparation of Fermented Broth

21-Day-old colonies of each S. sanghuang 38847 strain on malt extract agar (MEA) medium in a 9-cm Petri dish were cut into a bottle with 200 ml distilled water and blended for 30 seconds to prepare a fungal inoculum for liquid fermentation. 10 ml fungal inoculum was added to a 500-ml flask containing 200 ml liquid cultural medium (30 g corn starch, 10 g corn steep liquor, 5 g yeast extract, and 2 g sea salt in 1 L distilled water, pH 6). The inoculated medium was incubated at 25° C. for two weeks on a rotary shaker at the speed of 100 rpm. A total 14 L fungal fermented broth was harvested and then filtrated to remove fungal mycelium to obtain a fermentation product.

### Example 3

### Extraction and Isolation

Each of the fermentation products of S. sanghuang strains obtained from Example 2 (14 L) was extracted with BuOH to yield a BuOH extract (11.6 g) and a H₂O-soluble fraction (22.7 g). The BuOH extract (11.6 g) was subjected to silica gel column chromatography, wherein (CH₂Cl₂/ethyl acetate: 1:1) was used as the primary elution solution and MeOH was used to gradually increase the eluent polarity (CH₂Cl₂/ethyl acetate:1:1→CH₂Cl₂/ethyl acetate/MeOH: 1:1:0.5→CH₂Cl₂/ethyl acetate/MeOH:1:1:1→MeOH to produce 17 fractions (Fraction Nos. 1 to 17). Fraction 1 was purified by preparative TLC (CH₂Cl₂/acetone:40:1) to afford two eluted fractions, wherein the first eluted fraction comprises Compound 1 (36.4 mg) and the second eluted fraction comprises Compound 2 (12.8 mg). Compound is a yellowish oil. Compound 2 is a colorless oil.

### Example 4

### Characterizations of Compounds 1 and 2

Compound 1

The structure of Compound 1 was determined as (E)-5-(2,2-dimethyl-6-methylenecyclohexyl)-3-methylpent-2-enoic acid (also named sanghuanglin) having the following structure:

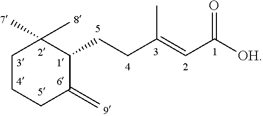

¹H- and ¹³C-NMR Spectroscopic Data for Compound 1 in CDCl₃ is shown in Table 2.

TABLE 2

| C | δ(H) (J, mult., Hz) | δ(C) |
|---|---|---|
| 1 | — | 172.0 (s) |
| 2 | 5.69 (br, q, J = 1.2) | 114.8 (d) |
| 3 | — | 164.0 (s) |
| 4 | 1.95 (m) | 39.8 (t) |
| | 2.15 (m) | |
| 5 | 1.56 (m) | 24.3 (t) |
| | 1.64 (m) | |
| Me-3 | 2.17 (d, J = 1.2) | 19.3 (q) |
| 1′ | 1.70 (dd, J = 12.0, 3.3) | 53.7 (d) |
| 2′ | — | 34.9 (s) |
| 3′ | 1.26 (dt, J = 12.6, 4.6, | 36.1 (t) |
| | Heq-β) | |
| | 1.48 (ddd, J = 12.6, 9.6, | |
| | 5.4, Hax-α) | |
| 4′ | 1.56 (m) | 23.6 (t) |
| 5′ | 2.04 (m) | 32.3 (t) |
| 6′ | — | 148.8 (s) |
| 7′ | 0.85 (s) | 26.3 (q) |
| 8′ | 1.21 (s) | 28.3 (q) |
| 9′ | 4.55 (br, d, J = 1.2) | 109.4 (t) |
| | 4.79 (br, sex, J= 1.2) | |

Assignments were done by HSQC, HMBC, and COSY experiments.

Compound 2

Compound 2 was determined as (+)-(2E,-4E)-5-((S)-2,2-dimethyl-6-methylenecyclohexyl)-3-methylpenta-2,4-dienoic acid (also called MDA) having the following structure:

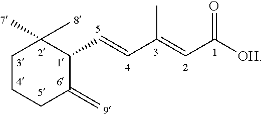

¹H- and ¹³C-NMR Spectroscopic Data for Compound 2 in CDCl₃ is shown in Table 3.

TABLE 3

| C | δ(H) (J, mult., Hz) | δ(C) |
|---|---|---|
| 1 | — | 171.4 |
| 2 | 5.75 (q, J = 0.9) | 117.1 |
| 3 | — | 155.0 |
| 4 | 6.14 (d, J = 15.6) | 135.3 |
| 5 | 6.32 (dd, J = 15.6, 9.6) | 137.3 |
| Me-3 | 2.32 (d, J = 0.9) | 14.3 |
| 1′ | 2.55 (d, J = 9.0) | 58.1 |
| 2′ | — | 35.7 |
| 3′ | 1.36 (m) | 39.0 |
| | 1.52 (m) | |
| 4′ | 1.61 (m) | 23.3 |
| 5′ | 2.06 (m,) | 34.4 |
| | 2.28 (m) | |
| 6′ | — | 149.4 |
| 7′ | 0.84 (s) | 23.6 |
| 8′ | 0.90 (s) | 29.4 |
| 9′ | 4.54 (br. s) | 109.0 |
| | 4.77 (br sex, J = 1.2) | |

Assignments were done by HSQC, HMBC, and COSY experiments.

### Example 5

### ER Total Activity Assay

Estrogen Receptor (ER) Model

ER is a ligand-activated enhancer protein that is a member of the steroid/nuclear receptor superfamily. ER binds to specific DNA sequences called estrogen response elements (EREs) (5′-GGTCAnnnTGACC-3′, SEQ ID NO: 7) with high affinity and transactivates gene expression in response to estradiol (E2) (Nucleic Acids Res. 2001 Jul. 15; 29(14): 2905-2919). Secreted alkaline phosphatase (SEAP) is a reporter widely used to study promoter activity or gene expression.

The 1 ml 0.05% trypsin-EDTA solution was added to 80% confluence (on Day 5, if cells were in log phase) of MCF-7 cells in a T75 flask at 37° C. for 5 minutes. After the cells were detached, 5 ml charcoal-treated FBS-containing medium were added to suspend the cells. The charcoal-treated FBS-containing PBS medium were added 2 ml cell suspensions to the final volume of 10 ml of a mixture. The mixture was dispensed into each well (100 μl/well, 2×10⁴ cells/well) of a 96-well plate by 8-channel pipette, and the 96-well plate was placed at 37° C. and 5% CO₂ overnight to obtain about 70% confluence.

To transfect cells to express the ERE, a transfection mixture was prepared. TransIT®-LT1 Transfection Reagent is a broad spectrum reagent that provides high efficiency plasmid DNA delivery in many mammalian cell types including primary cells. Specifically, 1 ml Opti-MEM®, 14 μl TransIT®-LT1 Transfection Reagent and 5 μg pERE-TA-SEAP were added into a 1.5 ml Eppendorf and let the transfection mixture stand for 15-30 minutes.

Transfection mixture containing TransIT-DNA was dispensed into each well (10 μl/well) of a 96-well plate, and the 96-well plate was placed at 37° C. and 5% CO₂ for 24 hours. The supernatants were removed from the 96-well plate using 8-channel pipette. 90 μl charcoal-treated FBS-containing medium (phenol red-free), optionally 0.1 nM E2 stimulator (depending on the purpose of screening), and 10 μl samples (E2, 38847 fermented broth, Compound 1 and Compound 2) were added to each well of the 96-well plate at 37° C., 5% CO₂ incubator for 48 hours.

Results

TABLE 4

| Samples | Related ER Activity^{a} |
|---|---|
| Medium Only | 1.00 ± 0.07 |
| 0.1 nM 17-β Estradiol (E2) | 10.99 ± 0.52 |
| 38847 fermented broth (1/10 v/v) | 10.19 ± 0.47 |

^{a}The relative estrogen receptor(ER) activity is expressed by the ratio between the activity of the sample of interest and the activity of untreated cells (Medium only)

As can be seen from Table 4, the fermented broths of 38847 fermented broth has a superior ER activity.

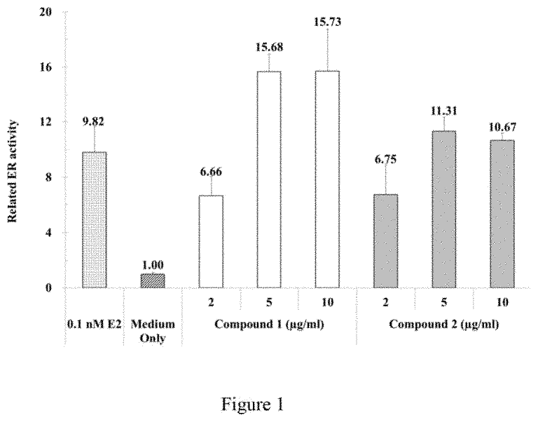

As shown in FIG. 1, Compounds 1 and 2 exhibited a significant ER activity at different concentrations (2 μg/ml, 5 μg/ml and 10 μg/ml). Surprisingly, it was found that Compounds 1 and 2 reached a superior ER activity over 17-β Estradiol (positive control).

### Example 6

### Evaluation of SERMs Activity

Cell Culture

CV-1 cells (Cercopithecus aethiops kidney cells) were grown at a confluence of 70-80% and were harvested in a cell suspension at a concentration of 5×10⁴ cell/ml. The cell suspensions were added to each well (100 μl/well) of 96-well plate and the 96-well plate was incubated at 37° C., 5% CO₂ incubator overnight.

Preparation of DNA Mixture for Transfection

500 μl jetPRIME® buffer. 5 μg pERE-TA-SEAP and 5 μg ERα DNA plasmid (or ERβ DNA plasmid) were mixed in 1.5 ml eppendorf, then 20 μl jetPRIME® Transfection Reagent and 1.5 ml medium were added. The DNA mixtures were added to each well of 96-well plate (20 μl) and the 96-well plate was incubated at 37° C., 5% CO₂ incubator for 4 hours.

Cell Harvest

The supernatant solutions were removed from each well of 96-well plate, then 90 μl charcoal-treated. FBS medium without phenol red were added to each well of 96-well plate. The samples to be tested were added to the wells of 96-well plate, which was incubated in 37° C., 5% CO₂ incubator for 48 to 72 hours. The cells were harvested for the subsequent reporter gene assay and cell toxicity assay.

Reporter Gene Assay

25 μl cell culture supernatants were collected from the 96-well plate and, then added to a new luminescence microplate, which was incubated at 65° C. water bath for 30 minutes to remove the activity endogenous alkaline phosphatase, and was placed on ice for 5 minutes and was centrifuged (1,000 rpm) for one minute. 25 μl PhosphaLight™ assay buffers were added at room temperature for 5 minutes and 25 μl CSPD (Disodium 3-(4-methoxyspiro {1,2-dioxetane-3,2′-(5′-chloro)tricyclo [3.3 1.13,7]decan}-4-yl phenyl phosphate)-containing Phospha-Light™ reaction buffers were added at room temperature for 20 minutes. The luminescence microplate was placed in Victor™ Light 1420 Luminescence Counter and the SEAP enzyme activities were measured.

Results

| | | ER Subtype Activity ^{a} | | |
|---|---|---|---|---|
| | Concentration | ER-α | ER-β | ER-β/ER-α |
| Medium Only | | 1.00 ± 0.22 | 1.00 ± 0.12 | 1.0 |
| 17-β Estradiol (E2) | 200 nM | 2.82 ± 0.79 | 3.70 ± 0.45 | 1.3 |
| Compound 1^{b} | 25 μg/mL | 1.21 ± 0.53 | 3.80 + 0.11 | 3.1 |
| Compound 2^{c} | 25 μg/mL | 1.16 ± 0.16 | 2.53 ± 0.77 | 2.2 |

^{a} The relative estrogen receptor(ER) activity is expressed by the ratio between the activity of the sample of interest and the activity of untreated cells (Medium only)
^{b}Compound 1 = sanghuanglin;
^{c}Compound 2 = (+)-(2E,4E)-5-((S)-2,2-dimethyl-6-methylenecyclohexyl)-3-methylpenta-2,4-dienoic acid (also called MDA)

17-β Estradiol (E2), as the positive control, showed an ER-β/ER-α ratio of 1.3. Surprisingly, compounds 1 and 2 showed an ER-β/ER-α ratio of 3.1 and 2.2, respectively. The results demonstrated that compounds 1 and 2 had a superior ER-β activity over ER-α activity, and thus had SERMs activity.

Numerous modifications and variations of the invention as set forth in the above illustrative examples are expected to occur to those skilled in the art Consequently, only such limitations as appear in the appended claims should be placed on the invention.

### REFERENCES

### Steroids. 2014, pii S0039-128X 14 00151.

### Tiosano et al, Reproductive Biology and Endocrinology 2014, 12: 97

Toxicol In Vitro, 2014 August; 28(5), pages 916-925
Fitoterapia 95 (2014), pages 93-101
Food Sci. Biotechnol, Vol. 17, No. 6, pages 1214-1220
Nucleic Acids Res. 2001 Jul 15; 29(14): pages 2905-2919

1. A method for the preparation of a liquid fermentate of Sanghuangporus comprising culturing Sanghuangporus in a broth.

2. A method for the preparation of a Sanghuangporus extract, comprising the steps of:
(a) obtaining a liquid fermentate of Sanghuangporus by culturing Sanghuangporus in a broth; and
(b) obtaining the Sanghuangporus extract by mixing the liquid fermentate of Sanghuangporus with alcohols.

3. A method for the preparation of 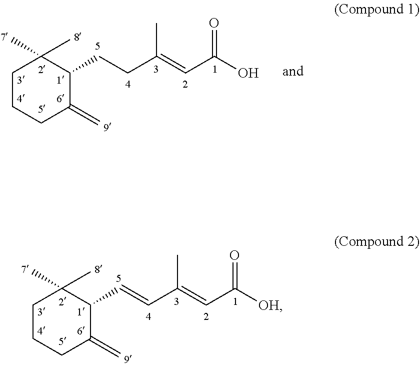
comprising the steps of
(a) obtaining a liquid fermentate of Sanghuangporus by culturing Sanghuangporus in a broth;
(b) obtaining an alcohol-soluble extract by mixing the liquid fermentate of Sanghuangporus with an alcohol;
(c) subjecting the alcohol-soluble extract to silica gel column chromatography;
(d) obtaining different eluates by eluting the column with elation solutions; and
(e) collecting a first eluted fraction and a second elated fraction by thin layer chromatography (CH₂Cl₂:acetone:40:1), wherein the first elated fraction comprises the Compound 1 and the second eluted fraction comprises the Compound 2.

4. The method of claim 1, wherein the Sanghuangporus is selected from the group consisting of S. sanghuang 38847 (DSMZ Accession No. DSM 32914).

5. The method of claim 2, wherein in step (b), the alcohol is selected from the group consisting of methanol, ethanol and butanol.

6. The method of claim 3, wherein in step (d), the column is elated with CH₂Cl₂/ethyl acetate (1:1).

7. The method of claim 3. wherein in step (d), the column is sequentially elated with CH₂Cl₂/ethyl acetate (1:1), CH₂Cl₂/ethyl acetate/MeOH (1:1:0.5) and CH₂Cl₂/ethyl acetate/MeOH (1:1:1).

8. The method of claim 3, after step (e), which further comprises a step of concentrating the elated fraction and/or drying the fraction to obtain a paste or solid fermentate.

9. A Sanghuangporus liquid fermentate obtained from the method of claim 1.

10. A Sanghuangporus extract obtained from the method of claim 2.

11. The Sanghuangporus liquid fermentate of claim 9, comprising

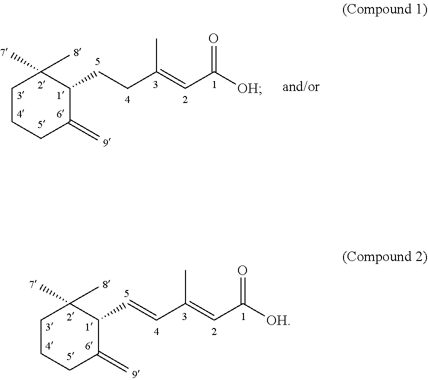

12. The Sanghuangporus extract of claim 10, comprising

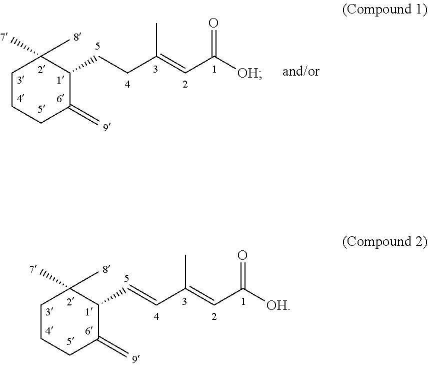

13. A pharmaceutical composition comprising the Sanghuangporus fermentate of claim 9 and a pharmaceutically acceptable carrier.

14. A method for preventing or treating an estrogen-dependent condition, disease, disorder, or syndrome in a subject in need thereof, comprising administering to the subject a therapeutically effective amount of the Sanghuangporus liquid fermentate of claim 9.

15. A method for preventing or treating an estrogen-dependent condition, disease, disorder, or syndrome in a subject in need thereof, comprising administering to the subject a therapeutically effective amount of a compound selected from

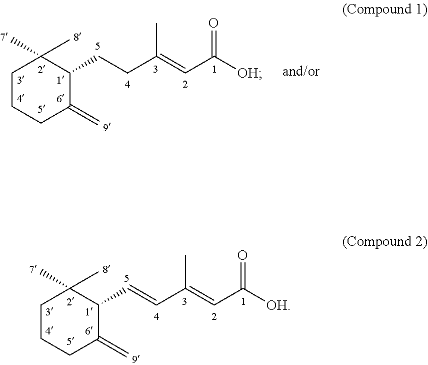

16. The method of claim 14, wherein the estrogen-dependent condition, disease, disorder, or syndrome is selected from the group consisting of mastodynia (breast pain/tenderness), breast fibroids, mammoplasia (breast enlargement), macromastia (breast hypertrophy), cardiovascular disease. stroke, gynecomastia, breast cancer, osteoporosis, precocious puberty in girls, melasma, menorrhagia, endometriosis, endometrial hyperplasia, adenomyosis, uterine fibroids, uterine cancers, ovarian cancer and hyperestrogenism.

17. The method of claim 14, wherein the liquid fermentate has an estrogen receptor (ER) activity and a selective estrogen receptor modulator (SERMs)

18. The method of claim 15, wherein the compounds 1 and 2 have an ER activity and a SERMs activity.

19. A method for screening a compound having an estrogen receptor (ER) activity comprising the steps of:
(a) providing transfected cells expressing an estrogen response elements (ERE) and a reporter gene;
(b) culturing the transfected cells in the presence of the compound, a positive control or a negative control; and
(c) measuring the activity of the reporter gene;
wherein the elevated activity of the reporter gene as compared to the negative control indicates that the compound has an ER activity, and wherein the positive control is

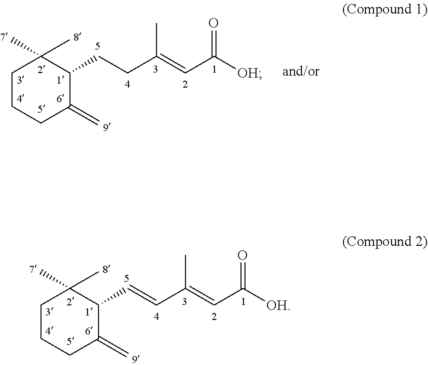

20. A pharmaceutical composition comprising the Sanghuangporus extract of claim 10 and a pharmaceutically acceptable carrier.
